(19) United States
(12) Patent Application Publication
(10) Pub. No.: US 2016/0228643 A1
(43) Pub. Date: Aug. 11, 2016

(54) SYRINGE AUGMENTATION DEVICE SYSTEMS AND METHODS

(71) Applicant: AIR-TITE PRODUCTS CO., INC., Virginia Beach, VA (US)

(72) Inventors: Paul Ellsworth Oberdorfer, Virginia Beach, VA (US); Forrest Ellingsworth, Hampton, VA (US)

(21) Appl. No.: 15/018,165

(22) Filed: Feb. 8, 2016

Related U.S. Application Data
(60) Provisional application No. 62/113,713, filed on Feb. 9, 2015.

(51) Int. Cl. A61M 5/20; A61M 5/31

(52) U.S. Cl. CPC A61M 5/2033; A61M 5/3137; A61M 2005/3139

(57) ABSTRACT

A syringe augmentation device and methods for using the same may be adapted to receive a disposable syringe. The syringe augmentation device may include a handle body having front and back members, and a syringe injector. The syringe injector may be configured to drive forward a ratchet shaft, which is attachable to an actuating horn. The syringe augmentation device may also include an attachment body configured to attach to the syringe injector. The attachment body may house a compression sleeve configured to receive a disposable syringe. When positioned within the compression sleeve, the actuating horn may abut a plunger of the syringe. In this configuration, as an operator squeezes the handle body, it may drive the ratchet shaft through the syringe injector. In turn, the ratchet shaft may direct the actuating horn against the syringe's plunger, causing the syringe to compress.

### CROSS REFERENCE TO RELATED APPLICATIONS

This application claims the benefit of U.S. Provisional Application No. 62/113,713, titled “SYRINGE AUGMENTATION DEVICE SYSTEMS AND METHODS,” filed Feb. 9, 2015 and which is fully incorporated by reference.

### FIELD OF THE INVENTION

The presently disclosed subject matter relates generally to systems and methods for a syringe augmentation device, particularly systems and methods for using an ergonomic syringe augmentation device with disposable syringes.

### BACKGROUND

Syringes are commonly used to administer injections, insert intravenous drugs into the bloodstream, apply compounds (e.g., glue, lubricants, caulking), and measure liquids. To reduce costs, particularly in the increasingly expensive healthcare field, reusable syringes are often used. One drawback of reusable syringes is that they require extensive cleaning to remove all biological and chemical contaminates prior to subsequent use. Improper cleaning can lead to serious complications in healthcare applications and inferior results in a laboratory environment. Creating a cleaning procedure, executing the procedure, and ensuring quality control can be very timely and expensive.

Sterile disposable syringes provide an alternative to reusable syringes without the associated cleaning expenses. While sterile disposable syringes can perform some of the same functions as traditional syringes and reusable syringes, they may have limitations. For example, sterile disposable syringes can be imprecise in repetitive applications, which could lead to complications and inferior results. They are also prone to produce fatigue in the hand after repeated operation, leading to costly work-related musculoskeletal disorders. Further, it may be difficult to control the pressure applied to disposable syringes during use, as conventional disposable syringes typically are constructed from less expensive parts and provide no visible pressure feedback.

Accordingly, there is a need for improved systems and methods to address the above mentioned deficiencies. Embodiments of the present disclosure are directed to these and other considerations.

### SUMMARY

Briefly described, embodiments of the presently disclosed subject matter relate to a syringe augmentation device and methods for using the same. Specifically, the syringe augmentation device may be adapted for use with one or more disposable syringes. In some embodiments, the syringe augmentation device may include a handle body having a front member and a back member and be configured for grasping and squeezing by an operator. At the top of the handle body, the syringe augmentation device may also include a syringe injector. The syringe injector may be configured to drive a ratchet shaft. The ratchet shaft may be connected to an actuating horn at one end and be configured to have its opposite, distal end extend, at least partially, through an opening in the syringe injector. The ratchet shaft may adjustably attach to the top of the back member and be configured to be ratcheted as the back member is squeezed towards the front member.

The syringe augmentation device may also include an attachment body configured to at least partially enclose the syringe injector. The attachment body may house a compression sleeve configured to receive a disposable syringe. When sitting in the compression sleeve, the actuating horn may abut a plunger of the syringe. In this configuration, as an operator squeezes the handle body, thereby forcing the front and back members towards one another, the back member may drive the ratchet shaft through the syringe injector. In turn, the ratchet shaft may pull the actuating horn forward against the syringe's plunger, causing the syringe to compress.

In some embodiments, the actuating horn may be a feedback actuating horn having a spring-loaded plunger, one or more pressure sensors, one or more feedback indicators, and a controller in communication with the sensors and indicators. The plunger may extend from the actuating horn to contact and compress the syringe as the handle body is squeezed. To monitor pressure as the syringe is compressed, the pressure sensors may measure the pressure applied to the plunger, and the controller may determine when the measured pressure exceeds one or more predetermined thresholds. In response to the determination that the measure pressure exceeds a predetermined threshold, the controller may direct one or more of the indicators to provide one or more of a visual and an auditory feedback signal, thereby providing feedback pressure to a user.

In some embodiments, the syringe augmentation device may be adaptable for use complete standard disposable syringes, such as, for example, 30 ml and 50 ml Soft-Ject® or Norm-Ject® disposable syringes. In other embodiments, the syringe augmentation device may include an LED pressure display that shows relative dispensing pressure.

### BRIEF DESCRIPTION OF THE DRAWINGS

FIG. 1 is an isometric view of a syringe augmentation device in accordance with some embodiments of the present disclosure.

FIG. 2 is top view of a syringe augmentation device in accordance with some embodiments of the present disclosure.

FIG. 3 is a side view of a syringe augmentation device in accordance with some embodiments of the present disclosure.

FIG. 4 is a rear view of a syringe augmentation device in accordance with some embodiments of the present disclosure.

FIG. 5 is an exploded view of a syringe augmentation device in accordance with some embodiments of the present disclosure.

FIG. 6 is an isometric view of a syringe augmentation device having pressure feedback indicators in accordance with some embodiments of the present disclosure.

FIG. 7 is top view of a syringe augmentation device having pressure feedback indicators in accordance with some embodiments of the present disclosure.

FIG. 8 is a side view of a syringe augmentation device having pressure feedback indicators in accordance with some embodiments of the present disclosure.

FIG. 9 is a rear left isometric view of a syringe augmentation device with a cross-sectional view of a feedback actuating horn of the device in accordance with some embodiments of the present disclosure.

FIG. 10 is a rear right isometric view of a syringe augmentation device with a cross-sectional view of a feedback actuating horn of the device in accordance with some embodiments of the present disclosure.

FIG. 11 is a flowchart of a method for augmenting a syringe with a syringe augmentation device in accordance with some embodiments of the present disclosure.

### DETAILED DESCRIPTION

The various embodiments of the presently disclosed subject matter are described with specificity to meet statutory requirements. However, the description itself is not intended to limit the scope of this patent. Rather, it has been contemplated that the claimed subject matter might also be embodied in other ways, to include different steps or elements similar to the ones described in this document, in conjunction with other present or future technologies.

It should also be noted that, as used in the specification and the appended claims, the singular forms “a,” “an” and “the” include plural references unless the context clearly dictates otherwise. References to a composition containing “a” constituent is intended to include other constituents in addition to the one named. Also, in describing the preferred embodiments, terminology will be resorted to for the sake of clarity. It is intended that each term contemplates its broadest meaning as understood by those skilled in the art and includes all technical equivalents which operate in a similar manner to accomplish a similar purpose.

Herein, the use of terms such as “having,” “has,” “including,” or “includes” are open-ended and are intended to have the same meaning as terms such as “comprising” or “comprises” and not preclude the presence of other structure, material, or acts. Similarly, though the use of terms such as “can” or “may” is intended to be open-ended and to reflect that structure, material, or acts are not necessary, the failure to use such terms is not intended to reflect that structure, material, or acts are essential. To the extent that structure, material, or acts are presently considered to be essential, they are identified as such.

It is also to be understood that the mention of one or more method steps does not preclude the presence of additional method steps or intervening method steps between those steps expressly identified. Moreover, although the term “step” may be used herein to connote different aspects of methods employed, the term should not be interpreted as implying any particular order among or between various steps herein disclosed unless and except when the order of individual steps is explicitly required.

The components described hereinafter as making up various elements of the disclosure are intended to be illustrative and not restrictive. Many suitable components that would perform the same or similar functions as the components described herein are intended to be embraced within the scope of the invention. Such other components not described herein can include, but are not limited to, for example, similar components that are developed after development of the presently disclosed subject matter.

To facilitate an understanding of the principles and features of the invention, various illustrative embodiments are explained below. In particular, the presently disclosed subject matter is described in the context of being a syringe augmentation device.

In some embodiments, the syringe augmentation device may include a handle body having a front member and a back member. The handle body may be configured for grasping and squeezing by an operator. At the top of the handle body, the syringe augmentation device may further include a syringe injector. The syringe injector may partially house a ratchet shaft, which may be attachable at one end to an actuating horn. The ratchet shaft may adjustably attach to the top of the back member and be configured to be ratcheted as the back member is squeezed towards the front member.

The syringe augmentation device may also include an attachment body configured to at least partially enclose or attach to the syringe injector. The attachment body may house a compression sleeve configured to receive a disposable syringe. When positioned within the compression sleeve, the actuating horn may abut or contact a syringe plunger of the syringe. In this configuration, as an operator squeezes the handle body, thereby forcing the front and back members towards one another, the back member may drive the ratchet shaft through the syringe injector. In turn, the ratchet shaft may pull the actuating horn forward against the syringe's plunger, causing the syringe to compress.

In some embodiments, the syringe augmentation device may be adaptable for use with 30 ml and 50 ml Soft-Ject® or Norm-Ject® disposable syringes. In other embodiments, the syringe augmentation device may include an LED pressure display that shows relative dispensing pressure. Embodiments of the disclosed tumbler will now be described with respect to the figures.

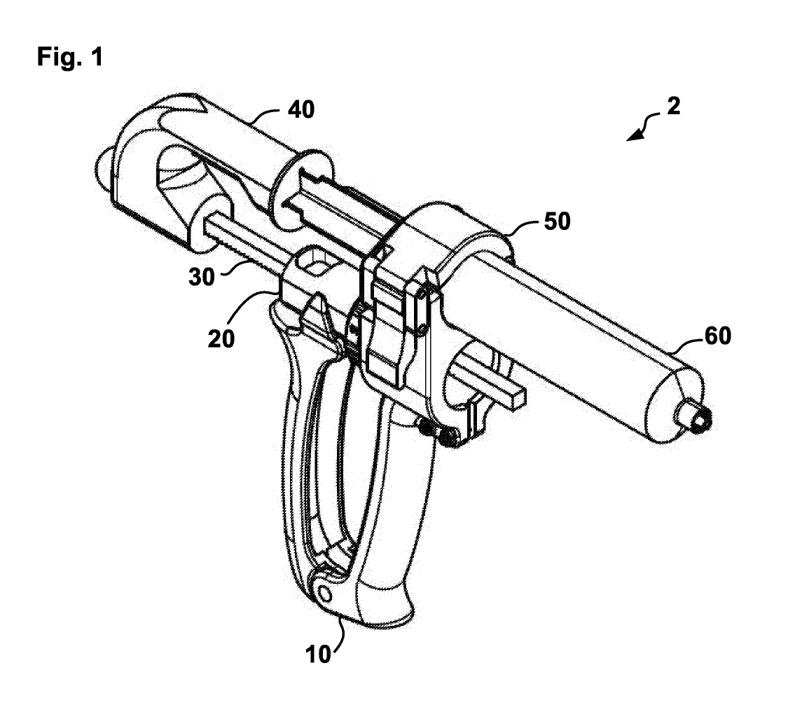

FIG. 1 is an exemplary embodiment of a syringe augmentation device 2. As shown, the syringe augmentation device 2 may include a handle body 10, a syringe injector 20, a ratchet shaft 30, an actuating horn 40, and an attachment body 50. The syringe augmentation device 2 may be configured to augment a disposable syringe 60 (e.g., 30 ml and 50 ml Soft-Ject® or Norm-Ject® disposable syringes). The syringe injector 20 may be attached to a top portion of the handle body 10, and configured to slidably receive the ratchet shaft 30. One end of the actuating horn 40 may attach to an end of the ratchet shaft 30, while the other end may abut against the syringe 60. Holding the syringe 60 in place, the attachment body 50 may be attachable to the syringe injector 20, and configured to slidably receive the syringe 60.

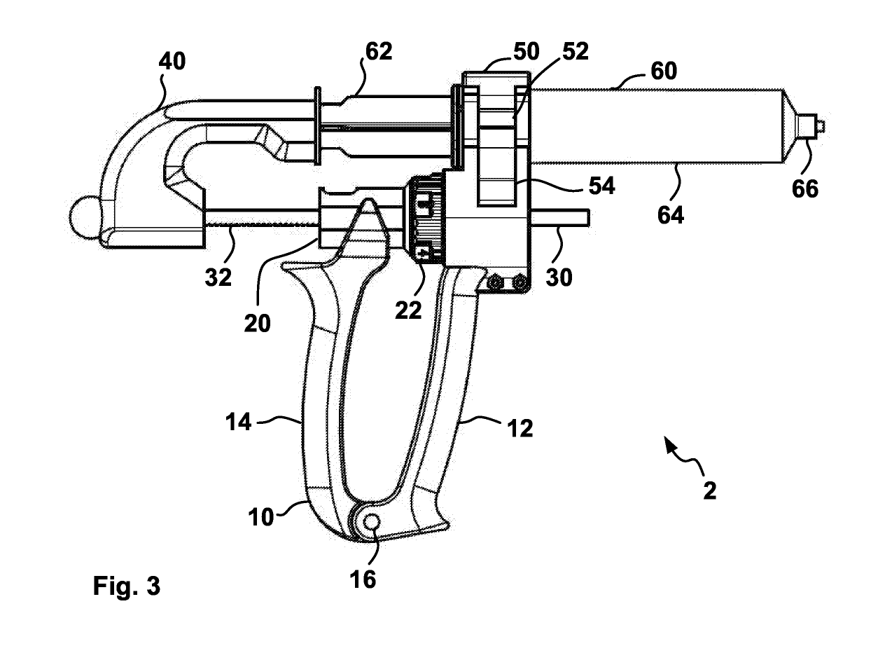

The handle body 10 may have a front member 12 and a back member 14. The bottom of the front member 12 may be pivotally joined to the bottom of the back member 14, as shown in FIGS. 1 and 3. For example, in some embodiments, this joint may include a joint pin 16 that slidably extends through holes in both members 12, 14. In other embodiments, one of the members 12, 14 may include one or more connectors that insert into holes in the opposing member. In this configuration, the front member 12 and the back member 14 may be configured to slide closer together when squeezed by an operator. In further embodiments, the front and back members 12, 14 may be pivotally connected at the top of the handle body 10.

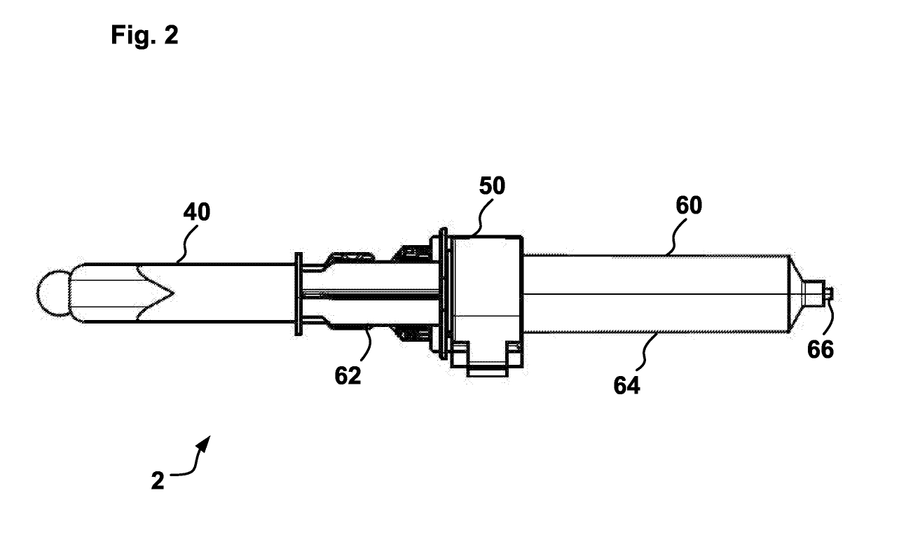

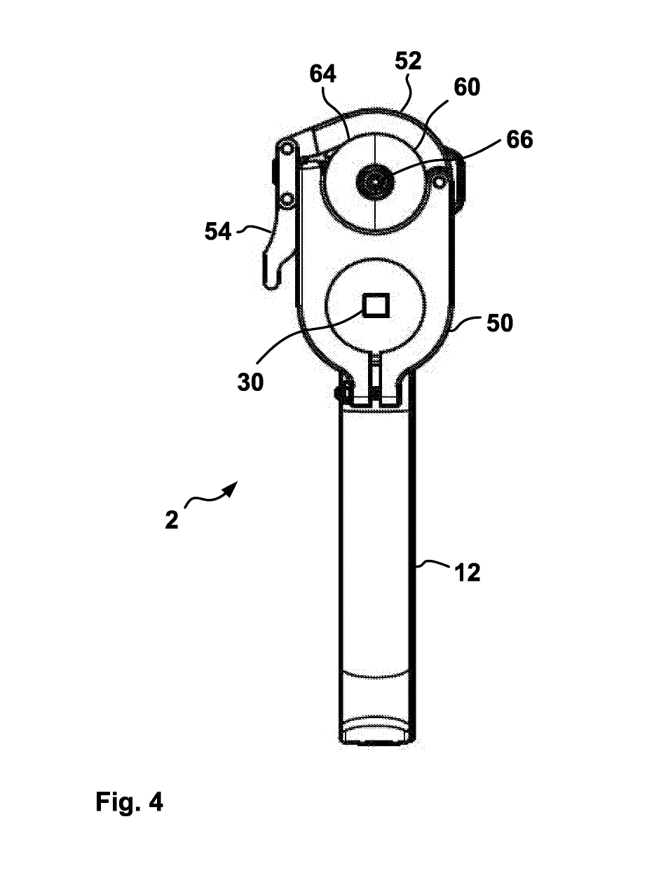

In some embodiments, the handle body 10 may be shaped for an operator's grip. For example, in one embodiment, the front member 12 may be smooth and slightly curved, as shown in FIG. 4, to receive the operator's fingers. In another embodiment, the front member 12 may have one or more indentations configured to receive the operator's fingers for improved grip. In some embodiments, the back member 14 may be shaped similarly to the back of a handgun, as shown in FIGS. 1 and 2, having a lip that extends outwardly at the top of the back member 14 for placement between the operator's thumb and index finger. The lip may help prevent the operator's hand from sliding upwardly into other components of the syringe augmentation device 2.

In some embodiments, the handle body 10 may be constructed from a metal, wood, or plastic. For example, in one embodiment, the handle body 10 may be constructed from pressure cast aluminum. The aluminum may advantageously keep the weight of the handle body 10 low, while providing adequate strength to withstand repeated squeezing. In another embodiment, the handle body 10 may be constructed from high temperature plastic, such as polysulfone, peek, torlon, which may provide the desired properties (e.g., strength, weight, etc.).

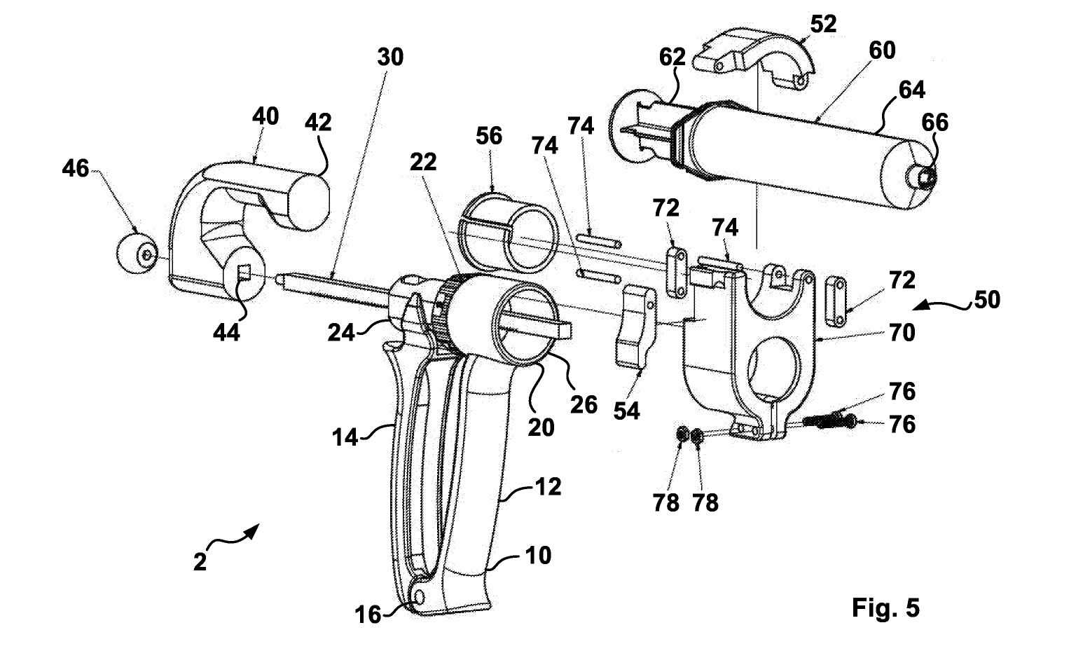

The syringe injector 20 may connect the tops of the front member 12 to the back member 14, as shown in FIGS. 1 and 3. In some embodiments, the syringe injector 20 may be removable from the handle body 10, and may be configured to readily attach and detach to the handle body 10 via connectors (e.g., bolts, screws, snaps, or other suitable connectors). Shown in more detail in FIG. 5, the syringe injector 20 may include a rotatable dial 22, a driver 24, and a drum 26. In some embodiments, the driver 24 may be attachable to the top of the back member 14, and the drum 26 may be attachable to the top of the front member 12. The driver 24 and drum 26 may be separated by the dial 22.

The dial 22 may be configured to rotate in a clockwise and/or counter-clockwise direction around the ratchet shaft 30 and relative to the drum 26. In some embodiments, rotating the dial 22 may alter the ratcheting of the ratchet shaft 30 with the syringe injector 20. For example, rotating the dial 22 in a clockwise direction may incrementally increase the amount at which each squeeze of the handle body 10 ratchets the actuating horn 40 forward. Similarly, rotating the dial 22 in a counter-clockwise direction may incrementally decrease the amount at which each squeeze of the handle body 10 ratchets the actuating horn 40 forward. The dial 22 may be shaped as a hollow ring of a diameter that matches the diameter of the drum 26. In other embodiments, the dial 22 may be sized to have a larger diameter than the drum 26, and may at least partially cover the drum 26. It is contemplated that the dial 22 may take on any shape as needed to fit with other components of the syringe augmentation device 2. The dial 22 may be constructed from a metal, wood, or plastic.

In some embodiments, the driver 24 may be attachable to the dial 22 and/or the drum 26, and configured to ratchet the ratchet shaft 30, thereby driving the actuating horn 40 forward. In one exemplary embodiment, the dial 22 may adjust how the driver 24 ratchets the ratchet shaft 30. The driver 24 may be adjustable in size and/or shape. In other embodiments, the driver 24 may be fixed in size and/or shape. The driver 24 may be constructed from a metal, wood, or plastic. For example, in some embodiments, the driver 24 may be constructed using stainless steel.

The drum 26 may be configured to house the dial 22 and the driver 24, and allow the ratchet shaft 30 to pass through it. In some embodiments, the drum 26 may form a hollow cylinder. It is contemplated, however, that the drum 26 may take on a variety of shapes as desired to fit with other components of the syringe augmentation device 2. The drum 26 may be constructed from a metal, wood, or plastic. In some embodiments, the drum 26 may be constructed using the same material as the handle body 10.

Partially disposed within the syringe injector 20, the ratchet shaft 30 may be attachable to a bottom end of the actuating horn 40. It is contemplated that the ratchet shaft 30 may be permanently connected to the actuating horn 40. Opposite the actuating horn 40, the ratchet shaft 30 may extend, at least partially, through the syringe injector 20. In some embodiments, the ratchet shaft 30 may include a plurality of teeth 32, as shown in FIG. 3. For example, in one embodiment, the bottom side of the ratchet shaft 30 may include the teeth 32. In other embodiments, however, the teeth 32 may be located on other or on multiple sides of the ratchet shaft 30.

In some embodiments, the dial 22 may adjust the number of teeth 32 that are ratcheted forward with each squeeze of the handle body 10. Following rotation of the dial 22, the driver 24 may adjust its attachment point(s) with the ratchet shaft 30 accordingly to change the number of teeth 32 that are ratcheted forward. There may be minimum and maximum settings of the number of teeth 32 that can be ratcheted per squeeze of the handle body 10. For example, the maximum setting may be predetermined to prevent the syringe 60 from being augmented too quickly.

In some embodiments, as shown in more detail in FIG. 5, the actuating horn 40 may include a hammer 42, a hole 44, and a plug 46. At the upper end of the actuating horn 40, the hammer 42 may be configured to push against the syringe 60. At the lower end of the actuating horn 40, the hole 44 may be sized and shaped to receive at least a portion of the ratchet shaft 30. On the back side of the lower end of the actuating horn 40, the plug 46 may be configured to seal hole 44.

The actuating horn 40 may be U-shaped, as shown in FIG. 3, in some embodiments. The longer the lower arm of the U-shaped actuating horn 40, the shorter the ratchet shaft 30 can be. In other embodiments, the actuating horn 40 may take on other shapes, such as a V-shape, C-shape, or half of a U-shape (e.g., without a second arm extending outwardly). The actuating horn 40 may be constructed from a metal, wood, or plastic. For example, in some embodiments, the actuating horn 40 may be constructed using stainless steel or aluminum. The selected material may have desired properties (e.g., weight, strength, durability, etc.).

At the top of the actuating horn 40, the hammer 42 may be sized and shaped to abut the syringe 60. The face of the hammer 42 may have a surface area sufficient to cover a rear surface area of the syringe 60, and may include gripping members (not shown) or other items to prevent slippage or uneven contact of the syringe 60. In some embodiments, the actuating horn 40 may form one continuous component, which includes the hammer 42. In other embodiments, the hammer 42 may be attachable to the actuating horn 40. In such embodiments, multiple hammers 42 of different sizes and/or shapes may be attachable to the actuating horn 40, with each size or shape being tailored to a different sized syringe 60 or a particular application.

Situated on the lower portion of the actuating horn 40 in some embodiments, the hole 44 may receive at least a portion of the ratchet shaft 30. The hole 44 may extend through the actuating horn 40 from the front side, facing the other components of the syringe augmentation device 2, to the back side. In other embodiments, the hole 44 may only extend from the front side through a portion of the actuating horn 40. The hole 44 may be sized and shaped to receive the ratchet shaft 30. For example, in one embodiment, the hole 44 may be a rectangular prism that is slightly larger than the rectangular prism shape of the ratchet shaft 30. In some embodiments, the hole 44 may provide some clearance area around the ratchet shaft 30 to allow for an adhesive, heat resistant, anti-friction, or corrosion resistant material to also be disposed within the hole 44. An adhesive may be used to help secure the ratchet shaft 30 within the hole 44 for a permanent or more stable connection. A heat-resistant or anti-friction material may help dissipate any heat or friction buildup between the actuating horn 40 and the ratchet shaft 30 during augmentation of the device 2. A corrosion resistant material may help prevent corrosion within the hole 44, which may be difficult to clean or repair without removal or replacement.

On the back side of the hole 44, the plug 46 may prevent any materials from entering the hole 44. The plug 46 may be sided and shaped to fit the hole 44. The plug 46 may also slidably receive a portion of the ratchet shaft 30 to help secure the ratchet shaft 30 within the hole 44. In some embodiments, the plug 46 may be constructed from a rubber or expandable plastic to help seal the back side of the hole 44.

In some embodiments, the attachment body 50 may be attachable to the syringe injector 20. For example, as shown in more detail in FIG. 5, the attachment body 50 may include a housing 70 configured to enclose the drum 26 of the syringe injector 20. The attachment body 50 may include a clamp top 52, a toggle handle 54, a compression sleeve 56, and the housing 70, as shown in FIGS. 3 to 5. Collectively, the top 52 and toggle handle 54 may secure the compression sleeve 56 on top of the housing 70. Once in position, the compression sleeve 56 may be configured to slidably receive the syringe 60.

In some embodiments, top 52 may pivotally connect to the housing 70 at one side. On the opposite side, the top 52 may pivotally connect to the toggle handle 54. Opposite the top 52, the toggle handle 54 may clamp down and hold a fixed position on its own or connect to the housing 70. The top 52 and/or the toggle handle 54 may be curved as needed to fit the compression sleeve 56 (or the surface of the syringe 60 if no compression sleeve 56 is present), and may have a width to cover a desired surface area of the compression sleeve 60. The free end of the toggle handle 54 may extend away from the housing 70 to more easily allow an operator to grasp and move the toggle handle 54.

The compression sleeve 56 may be shaped as desired to fit around the syringe 60. For example, in one embodiment, the compression sleeve 56 may be cylindrical and extend a portion of the length of the syringe 60. Seated between the syringe 60 and the attachment body 50, the compression sleeve 56 may snugly hold the syringe 60 in place and/or protect the syringe from forces, including vibrations of the syringe augmentation device 2 during use. Accordingly, the compression sleeve 56 may prevent the syringe 60 from undesirably falling out of the syringe augmentation device, which could result in damage of the syringe 60 or the patient undesirably being punctured by the syringe 60, while allowing an operator to insert and remove the syringe 60. In some embodiments, the compression sleeve 60 may include one or more gaps that allow the compression sleeve 56 to compress as needed. The compression sleeve 56 may be constructed from a metal, wood, or plastic. For example, in one embodiment, the compression sleeve 56 may be constructed using an elastic material, which advantageously allows the compression sleeve to expand or compress as needed during use.

The housing 70 may be shaped to at least partially cover the drum 26 of the syringe injector 20, and slidably receive the syringe 60. In some embodiments, the housing 70 may include one or more toggle lock bars 72, one or more pins 74, and a plurality of bolts 76 and nuts 78, as shown in FIG. 5. The toggle bars 72 and pins 74 may pivotally connect the housing 70, top 52, and toggle handle 54 to one another. It is contemplated that other connectors may be used to connect (pivotally or statically) the housing 70, top 52, and toggle handle 54. At the bottom of the housing, the bolts 76 may extend through holes defined by the housing 70 to connect the housing 70 to itself around the drum 26. The nuts 78 may be fasted to the threaded end of the bolts 76 after the bolts 76 are disposed through the housing 70. It is contemplated that any connector may be used to connect the housing 70. Further, the housing 70 may be designed such that it is expandable or otherwise grips the drum 26 without requiring connectors. In other embodiments, the bolts 76 and nuts 78 may connect the housing 70 together at another point on the housing 70.

The syringe 60 may be a complete standard disposable syringe, in some embodiments. As used herein, a complete standard disposable syringe includes conventional disposable syringes (e.g., 30 ml and 50 ml Soft-Ject® or Norm-Ject® disposable syringes) that have not been modified or had any components or portions removed outside of swapping between delivery members (e.g., a needle, a nozzle, an outlet, etc.). Shown in more detail in FIG. 2, the syringe 60 may include a plunger 62 configured to slide into and out of a cylindrical chamber 64 to force fluid contained within the chamber 54 out into a delivery member 66 (e.g., a nozzle, a needle, an outlet, etc.). In some embodiments, as shown in FIG. 5, the plunger 62 of the syringe 60 may abut against the hammer 42 of the actuating horn 40, and the chamber 64 may be partially housed within the compression sleeve 56 of the attachment body 50. In this configuration, the actuating horn 40 may be configured to direct the plunger 62 to slide into the chamber 64, causing the syringe 60 to compress and direct at least a portion of the fluid contained within the chamber 64 to exit the delivery member 66 of the syringe 60. The syringe 60 may be of any size and/or shape and the components of the syringe augmentation device 2 may be tailored to securely fasten and hold the syringe 60.

The disclosed syringe augmentation device 2 may be used with any disposable syringe for many different applications, including in a healthcare environment. In use, an operator may hold the handle body 10 with one or both hands. To insert the syringe 60 within the syringe augmentation device 2, the operator must first adjust the ratchet shaft 30 away from the syringe injector 20 to provide sufficient clearance between the actuating arm 40 and the plunger 62 of the syringe 60 so the syringe 60 can slide into the housing 70. The operator may then insert the chamber 54 of the syringe 60 into the compression sleeve 56 from behind, and slide the actuating arm 40 forward until it abuts the outer surface of the plunger 62. Optionally, in some embodiments, the syringe 60 may be placed within the compression sleeve 56 prior to insertion into the housing 70, and then the syringe 60 with the compression sleeve 56 may be inserted into the housing 70.

After the placement of the syringe 60 within the housing 70 and slidably engaging the hammer 42 against the plunger 62, the operator may adjust the dial 22 to a desired setting. In some embodiments, the operator may rotate the dial 22 clockwise to increase the ratcheting of the ratchet shaft 30 per squeeze of the handle body 10. For example, the operator may increase the ratcheting such that the ratchet shaft 30 moves forward three teeth 32 per squeeze of the handle body 10 instead of a default of moving forward one tooth 32 per squeeze. Similarly, the operator may rotate the dial 22 counter-clockwise to incrementally decrease the ratcheting per squeeze of the handle body 10. When the dial 22 is adjusted, the driver 24 correspondingly adjusts to drive the appropriate number of teeth 32 forward.

After adjusting the dial 22, the operator can augment the syringe and administer a contained fluid (e.g., a medical fluid or drug) to a patient or laboratory container. The operator may squeeze the handle body 10, thereby pushing the back member 14 forward with the operator's palm and/or pulling the front member 12 backwards with the operator's finger(s). This squeezing of the handle body 10 may direct the ratchet shaft 30 forward, thereby causing it to incrementally ratchet. As the ratchet shaft 30 moves forward, it pulls the actuating horn 40 forward with it. The forward movement of the actuating horn causes the hammer 42 to push against the plunger 62, thereby causing the plunger 62 to slide into the chamber 64. When the plunger 62 has sufficiently slid into the chamber 64, at least a portion of the fluid contained within the chamber 64 may be directed to exit through the delivery member 66. The syringe augmentation device 2 may continue to incrementally ratchet and direct the fluid to exit through the delivery member 66. Optionally, in some embodiments, the syringe augmentation device 2 may include a pressure feedback display (not shown) in communication with a pressure sensor coupled to the syringe 60 (e.g., at chamber 64, delivery member 66, etc.). The pressure feedback display may provide an operator with the current pressure in the syringe 60 to help maintain a desired pressure and/or alert the operator to unexpected or undesirable operation.

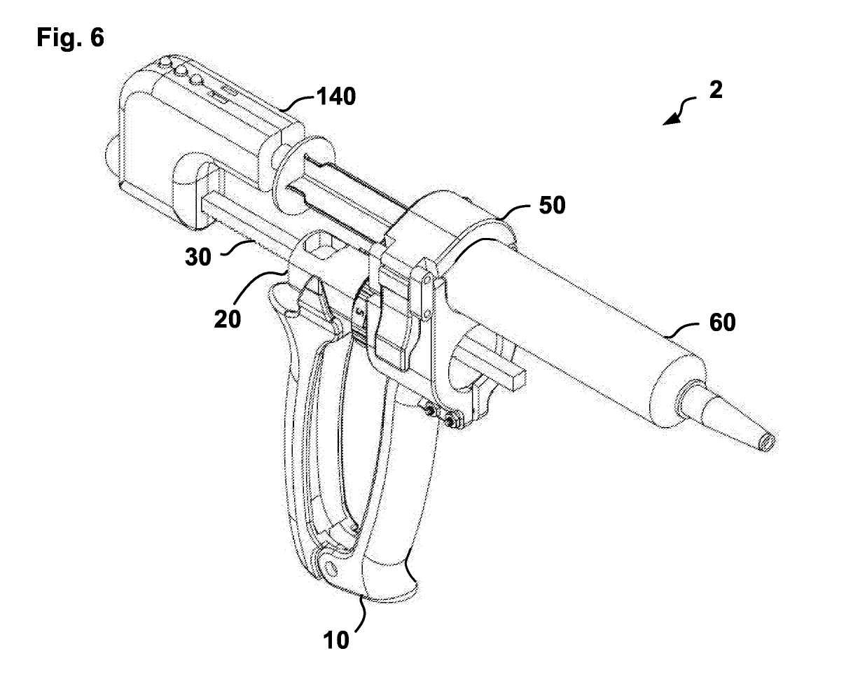

FIGS. 6-10 provide other exemplary embodiments of the syringe augmentation device 2, which may be configured to provide pressure feedback during use (e.g., as the syringe 60 is compressed). The pressure feedback can be critical, particularly for pressure sensitive delivery and with pressure sensitive medicines. As shown in FIG. 6, the syringe augmentation device 2 may include a feedback actuating horn 140 in lieu of actuating horn 40.

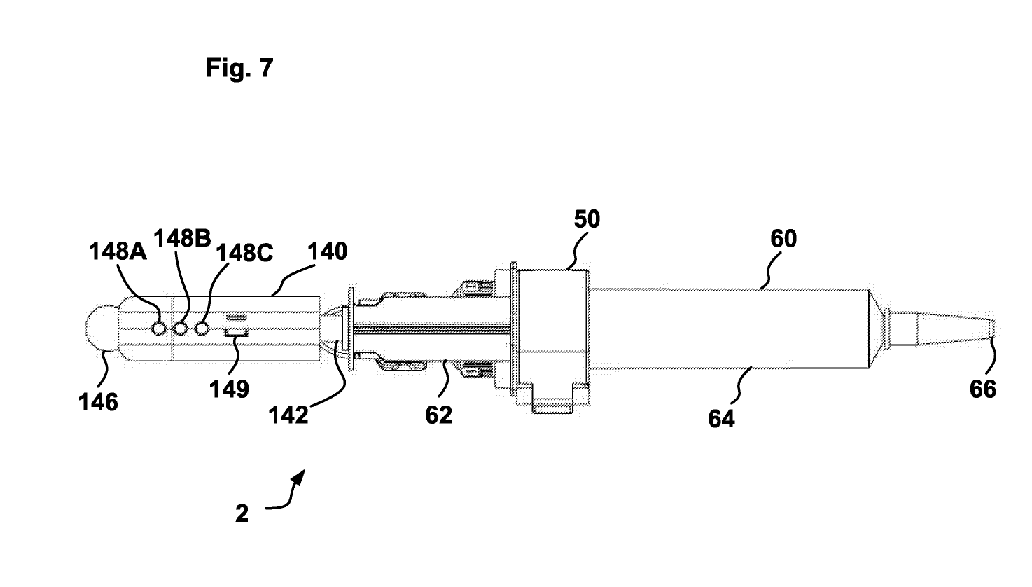

Shown in more detail in FIG. 7, the feedback actuating horn 140 may include a plunger 142, a hole 144, a plug 146, one or more feedback indicators 148A-C, and attachment clips 149. At the upper end of the actuating horn 40, the plunger 142 may be configured to push against the syringe 60. At the lower end of the feedback actuating horn 140, the hole 144 may be sized and shaped to receive at least a portion of the ratchet shaft 30. On the back side of the lower end of the feedback actuating horn 140, the plug 146 may be configured to seal hole 144.

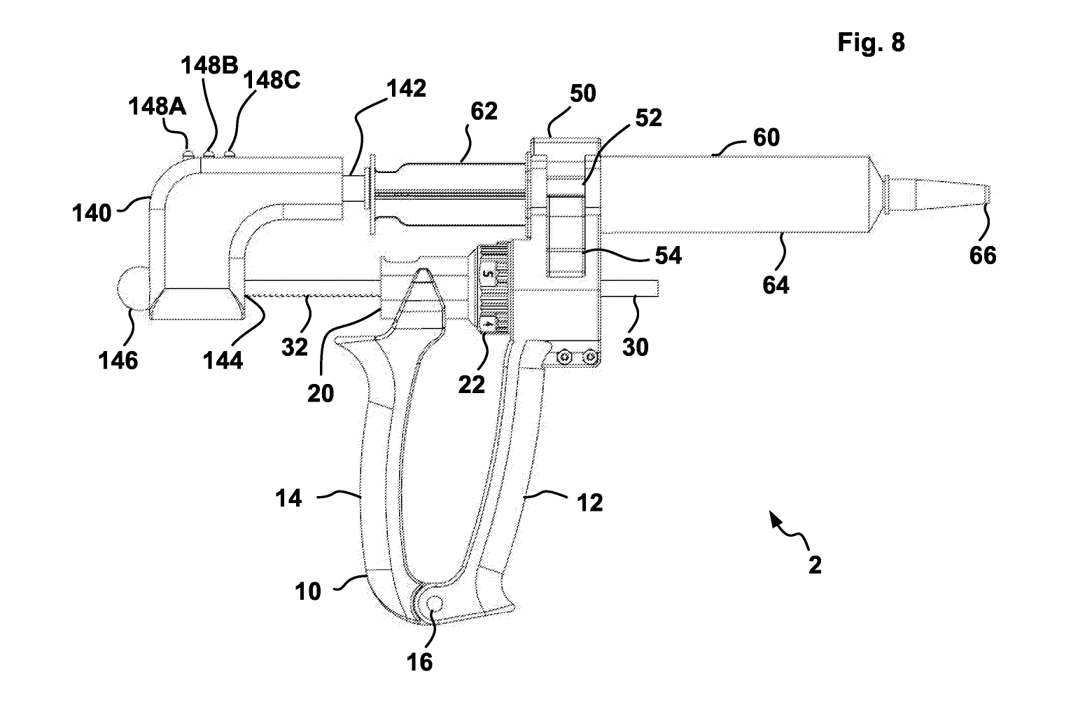

In some embodiments, the feedback actuating horn 140 may be half U-shaped (without a second arm extending outwardly), as shown in FIG. 8, which may be easier to manufacture than other shapes. In other embodiments, the feedback actuating horn 140 may be U-shaped like the actuating horn 40 shown in FIG. 3, which allows a shorter ratchet shaft 30 to be used. In further embodiments, the feedback actuating horn 140 may take on other shapes, such as a V-shape, C-shape, or an L-shape. The feedback actuating horn 140 may be constructed from a metal, wood, or plastic. For example, in some embodiments, the feedback actuating horn 140 may be constructed using stainless steel or aluminum. The selected material may have desired properties (e.g., weight, strength, durability, etc.).

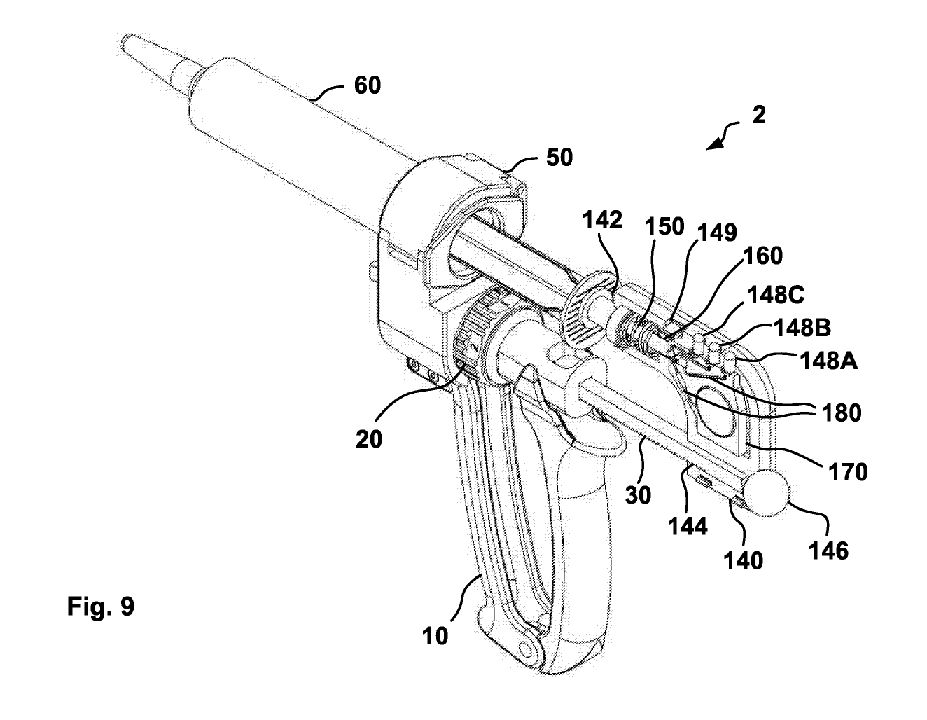

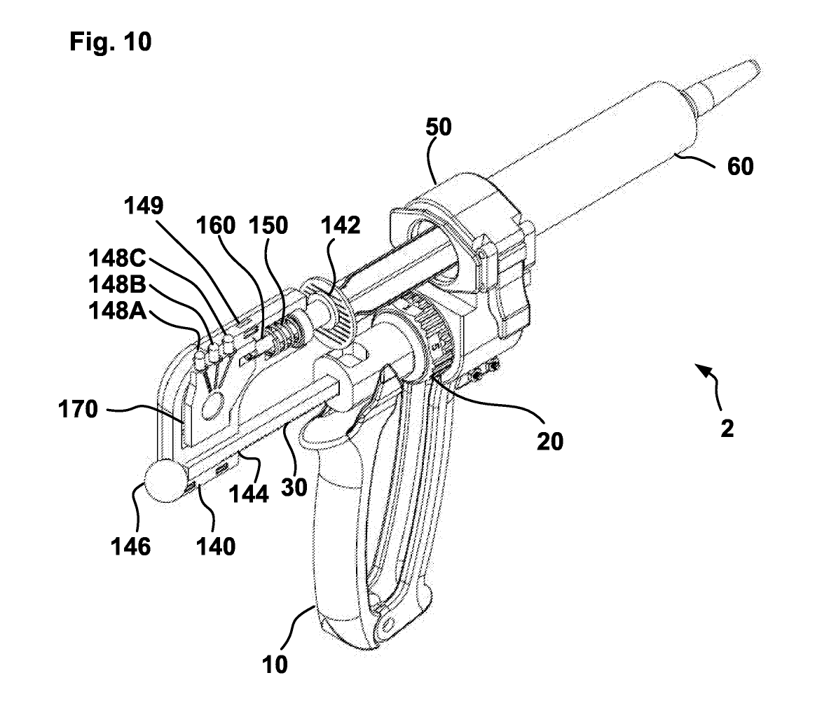

At the top of the feedback actuating horn 140, the plunger 142 may be sized and shaped to abut the syringe 60. In some embodiments, as shown more clearly in the partial cross-sectional views in FIGS. 9 and 10, the plunger 142 may be a spring-loaded plunger 142 having a spring 150. In some embodiments, the spring 150 may be a coil spring, though it is contemplated that springs of any type, size, and compression rate may be used. In some embodiments, the face of the plunger 142 may have a smaller surface area than the rear surface area of the syringe, as shown in FIGS. 7, 9, and 10. In other embodiments, the face of the plunger 142 may have a surface area sufficient to cover a rear surface area of the syringe 60. Optionally, the contact surface of the plunger 142 may include gripping members (not shown) or other items to prevent slippage or uneven contact of the syringe 60. It is also contemplated that the syringe 60 may include one or more gripping members, and the contact surface of the plunger 142 may have one or more mating gripping members to match a pattern of those on the syringe 60. In some embodiments, the feedback actuating horn 140 may form one continuous component, which includes the plunger 142. In other embodiments, the plunger 142 may be attachable to the feedback actuating horn 140. In such embodiments, multiple plungers 142 of different sizes and/or shapes may be attachable to the feedback actuating horn 140, with each size or shape being tailored to a different sized syringe 60 or a particular application.

Situated on the lower portion of the actuating horn 40 in some embodiments, the hole 144 may receive at least a portion of the ratchet shaft 30, as previously explained with respect to the hole 44 of the actuating horn 40. Similarly, on the back side of the hole 144, the plug 146 may prevent any materials from entering the hole 144, as previously explained with respect to the plug 46 of the actuating horn 40.

Shown on top of the feedback actuating horn 140 for illustrative purposes in FIGS. 7-10, the feedback indicators 148A-C may be positioned in an easily viewable position (from a user's perspective) during use anywhere on the syringe augmentation device 2. In this manner, the feedback indicators 148A-C may provide pressure feedback to the user in a manner than can be quickly processed and, if needed, acted upon during use. In some embodiments, the feedback indicators 148A-C may include one or more lights, and the lights may be configured to illuminate in different colors than one another (e.g., feedback indicators 148A, 148B, and 148C could illuminate in green, yellow, and red, respectively). In other embodiments, the feedback indicators 148A-C may include one or more speakers configured to provide auditory signals to a user, and the auditory signals may differ from one another (e.g., feedback indicators 148A, 148B, and 148C could each produce a different type of auditory signal, such as a beep, chirp, chime, etc., and/or a different volume of auditory signal). In further embodiments, the feedback indicators 148A-C may include one or more vibrating devices configured to provide tactile feedback signals to a user, and the tactile signals may differ from one another (e.g., single vibration, heartbeat pulse vibration, rapid pulse vibration, etc.). In this manner, the vibration frequency and/o may continue to increase It is contemplated that the feedback indicators 148A-C may be configured to provide other types of feedback or multiple types of feedback to the user.

Also shown on top of the feedback actuating horn 140 for illustrative purposes in FIGS. 7, 9, and 10, the attachment clips 149 may be configured to attach together to hold the assembled feedback actuating horn 140 together. The attachment clips 149 may be selectively dis-attached to provide access to one or more inner components of the feedback actuating horn 140. It is contemplated that the attachment clips 149 may include any type of attachable members (e.g., clips, snaps, hooks, etc.).

Disposed within the feedback actuating horn 140, as shown in FIGS. 9 and 10, the inner components may include the spring 150, one or more pressure sensors 160, a controller 170, and electrical wiring 180. The one or more sensors 160 may be configured to measure a pressure applied to the plunger 142. The controller 170, which is in communication with the one or more sensors 160 and the one or more feedback indicators 148A-C via the electrical wiring 180, may be configured to determine when the measured pressure exceeds a predetermined threshold and, in response, direct the one or more indicators 148A-C to provide one or more feedback signals (e.g., illuminate in a particular color, provide a particular auditory signal, etc.) to provide feedback to the user of the pressure within the syringe 60.

The one or more pressure sensors 160 may include electronic pressure sensors (e.g., piezoelectric pressure sensors), and may or may not include a shaft within the plunger 142. In other embodiments, any type of sensor may be used to measure pressure.

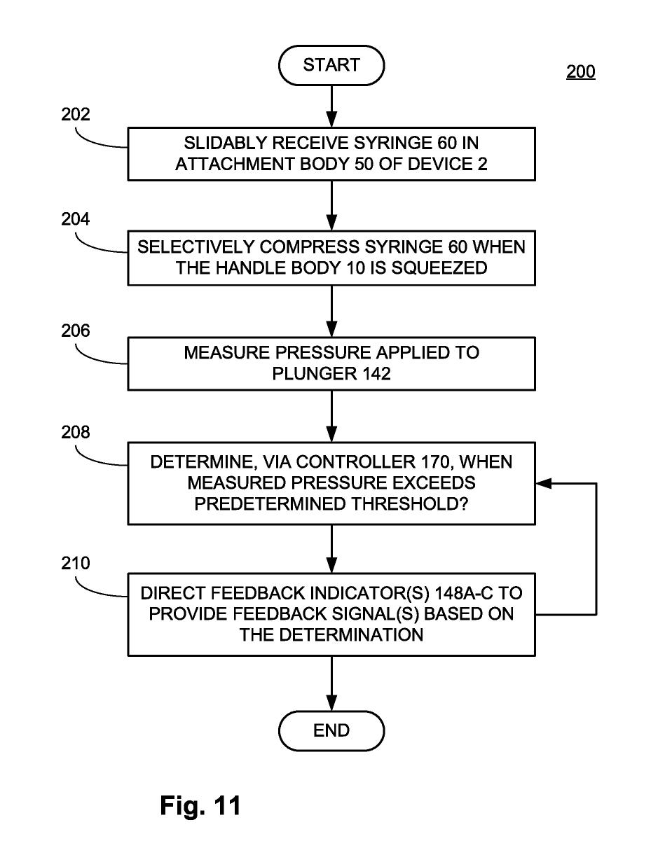

The controller 170 may have one or more processors having memory storing instructions, and the processor(s) may be configured to execute the instructions to perform one or more methods disclosed herein. For example, FIG. 11 provides an exemplary method of augmenting a syringe in accordance with some embodiments. The syringe augmentation device 2 may slidably receive the syringe 60 (e.g., within the compression sleeve 56 of the attachment body 50) at step 202 and may selectively compress the syringe 60 (e.g., by driving the ratchet shaft 30, and thus, the feedback actuating horn 140 and the plunger 142, forward) when the handle body 10 is squeezed at step 204. Meanwhile, the one or more pressure sensors 160 may measure the pressure applied to the plunger 142, at step 206. The controller 170 may then determine when the measured pressure exceeds one or more predetermined thresholds at step 208, and in response, direct the one or more feedback indicators 148A-C to provide one or more feedback signals based on the determination at step 210.

The controller 170 may have a plurality of predetermined pressure thresholds stored therein, and these predetermined pressure thresholds may be adjustable or input by the user. For example, the controller 170 may determine that the measured pressure exceeds a first predetermined threshold (e.g., 5 psi) and in response, may direct the first feedback indicator 148A to illuminate green. As the user continues to squeeze the handle body 10, the syringe augmentation device 2 continues to iteratively compress the syringe 60 in increments based on the ratcheting of the ratchet shaft 30. When the controller 170 determines that the measured pressure exceeds a second predetermined threshold (e.g., 8 psi), it may direct the second feedback indicator 148B to illuminate yellow. As the syringe augmentation device 2 continues to compress the syringe 60, thereby increasing the pressure applied to the plunger 142, the controller 170 may determine that the measured pressure exceeds a third predetermined threshold (e.g., 10 psi) and in response, may direct the third feedback indicator 148C to illuminate red. Due to the positioning of the feedback indicators 148A-C, the user may be able to easily view the pressure in the syringe 60 during compression, and thus can avoid applying too much pressure on a standard disposable syringe, which may disrupt proper delivery or damage the contents of the syringe 60. The predetermined pressure threshold(s) may be set based on the pressure sensitivity of the contents of the syringe 60. In some embodiments, the spring 150 may be selected of a particular size and/or compression rate to correspond with the predetermined pressure thresholds (e.g., the predetermined pressure thresholds may correspond to when the spring 150 is unloaded, partially loaded, and fully loaded, respectively).

It is also contemplated that, even though the feedback indicators 148A-C may be positioned such that they are easily visible, it may be helpful to provide the user with auditory feedback (alone or in addition to visual feedback) for certain applications. For example, in some embodiments, the controller 170 may direct the feedback indicator 148C to provide an auditory signal (e.g., a beep) in addition to illuminating red when the controller determines that the measured pressure exceeds the third predetermined threshold. In other embodiments, the controller 170 may direct the feedback indicator 148C to provide a tactile signal (e.g., a pulsed vibration) in addition to or in lieu of illuminating red and/or beeping. These exemplary non-visual feedback signals may help alert a user that a maximum pressure threshold is being approached even when the feedback indicators 148A-C are out of the user's view. While discussed with respect to feedback indicator 148C, one or more of the auditory and tactile feedback signals may also be present in feedback indicators 148A and 148B. Further, it is contemplated that the controller 170 and the feedback indicators 148A-C may be configured to provide other types of pressure feedback to the user, and the controller 170 may be configured to communicate with external devices (e.g., an external display) and display and/or log the measured pressure during compression of the syringe 60.

The syringe augmentation device 2 may provide several advantages over existing designs. For example, the incremental ratcheting of the syringe augmentation device 2 may allow for a precise, repetitive action of compressing the syringe 60. This repetitive, even compression may produce superior or more exact results. Also, by allowing an operator to squeeze the handle body 10 in lieu of pressing the plunger 62 down directly, the syringe augmentation device 2 may be less likely to cause fatigue or musculoskeletal disorders. Further, the syringe augmentation device 2 may be used in conjunction with any syringe 60, including conventional disposable syringes. In operation, the syringe augmentation device 2 may provide a disposable syringe, which are often difficult to precisely control as they are manufactured with less expensive parts, with improved control and drug delivery. By being configured to augment a complete standard syringe, the syringe augmentation device 2 avoids unnecessary time and expense of customizing each disposable syringe for device compatibility. Further, the pressure feedback display may alert an operator to easily observe and, if necessary, act upon the pressure of the syringe 60 to achieve desired results.

While the present disclosure has been described in connection with a plurality of exemplary aspects, as illustrated in the various figures and discussed above, it is understood that other similar aspects can be used or modifications and additions can be made to the described aspects for performing the same function of the present disclosure without deviating therefrom. For example, in various aspects of the disclosure, methods and compositions were described according to aspects of the presently disclosed subject matter. However, other equivalent methods or composition to these described aspects are also contemplated by the teachings herein. Therefore, the present disclosure should not be limited to any single aspect, but rather construed in breadth and scope in accordance with the appended claims.

What is claimed is:

1. A syringe augmentation device for augmenting a complete standard disposable syringe, the syringe augmentation device comprising:
a squeezable handle body having a front member and a back member, the front and back members being configured to move relative to one another;
a syringe injector attachable to the front and back members of the handle body;
an attachment body having a first cavity and a second cavity, the first cavity being configured to at least partially house the syringe injector and the second cavity being configured to at least partially house the syringe;
a ratchet shaft in mechanical communication with the syringe injector, the ratchet shaft being configured to be ratcheted as the back member is squeezed towards the front member; and
an actuating horn attachable to the ratchet shaft, the actuating horn being configured to at least partially compress the syringe as the ratchet shaft is ratcheted.

2. The syringe augmentation device of claim 1, wherein the attachment body further comprises a compression sleeve configured to receive a cylindrical chamber of the syringe, the compression sleeve being configured to maintain the position of the cylindrical chamber of the syringe when the syringe is compressed.

3. The syringe augmentation device of claim 1, wherein the actuating horn comprises a spring-loaded plunger, the plunger being configured to contact a syringe plunger of the syringe when the syringe is compressed.

4. The syringe augmentation device of claim 3, further comprising one or more pressure sensors configured to measure a pressure applied to the plunger.

5. The syringe augmentation device of claim 4, further comprising:
a controller having one or more processors, the controller being housed within the syringe augmentation device, wherein the controller is configured to determine when the measured pressure exceeds a predetermined threshold; and
one or more feedback indicators in communication with the controller, the one or more feedback indicators being configured to provide one or more of at least one visual feedback signal and at least one auditory feedback signal based on the determination.

6. The syringe augmentation device of claim 5, wherein the one or more feedback indicators comprise one or more lights, and the controller is configured to direct the one or more lights to illuminate based on the determination.

7. The syringe augmentation device of claim 5, wherein the one or more feedback indicators comprise one or more speakers, and the controller is configured to direct the one or more speakers to provide an auditory signal based on the determination.

8. A syringe augmentation device for augmenting a disposable syringe, the syringe augmentation device comprising:
a squeezable handle body having a front member and a back member, the front and back members being configured to move relative to one another;
a syringe injector attachable to the front and back members of the handle body;
an attachment body having a first cavity and a second cavity, the first cavity being configured to at least partially house the syringe injector and the second cavity being configured to at least partially house the syringe;
a ratchet shaft in mechanical communication with the syringe injector, the ratchet shaft being configured to be ratcheted as the back member is squeezed towards the front member;
an actuating horn attachable to the ratchet shaft;
a spring-loaded plunger extending from an end of the actuating horn, the plunger being configured to at least partially compress the syringe as the ratchet shaft is ratcheted;
one or more pressure sensors disposed within the actuating horn, the one or more pressure sensors being configured to measure a pressure applied to the plunger;
one or more feedback indicators attached to the actuating horn;
a controller in electrical communication with the one or more pressure sensors and the one or more feedback indicators, the controller being configured to determine when the measured pressure exceeds a predetermined threshold and to direct the one or more feedback indicators to illuminate based on the determination.

9. The syringe augmentation device of claim 8, wherein the disposable syringe is a complete standard disposable syringe having a cylindrical chamber and a syringe plunger.

10. The syringe augmentation device of claim 9, wherein the attachment body further comprises a compression sleeve configured to receive the cylindrical chamber of the syringe, the compression sleeve being configured to maintain the position of the cylindrical chamber of the syringe when the syringe is compressed.

11. The syringe augmentation device of claim 8, wherein the controller is further configured to determine when the measured pressure exceeds one or more of at least a first predetermined threshold and second predetermined threshold different from the first predetermined threshold.

12. The syringe augmentation device of claim 11, wherein the one or more feedback indicators comprises at least a first feedback indicator and a second feedback indicator, the first feedback indicator being configured to illuminate a first color and the second feedback indicator being configured to illuminate a second color different from the first color.

13. The syringe augmentation device of claim 11, wherein the one or more feedback indicators comprises a single feedback indicator, and the controller is further configured to direct the single feedback indicator to illuminate a first color based on the determination that the measured pressure exceeds the first predetermined threshold and to illuminate a second color based on the determination that the measured pressure exceeds the second predetermined threshold, the second color differing from the first color.

14. The syringe augmentation device of claim 11, wherein the controller is further configured to direct one or more auditory feedback indicators to provide a first auditory signal based on the determination that the measured pressure exceeds the first predetermined threshold and to provide a second auditory signal based on the determination that the measured pressure exceeds the second predetermined threshold, the second auditory signal differing from the first auditory signal.

15. A method of augmenting a complete standard disposable syringe, the method comprising:
slidably receiving the syringe in an attachment body of a syringe augmentation device;
selectively compressing the syringe via a spring-loaded plunger of the syringe augmentation device when a handle body of the syringe augmentation device is squeezed;
measuring a pressure applied to the plunger of the syringe augmentation device;
determining when the measured pressure exceeds a predetermined threshold;
illuminating one or more feedback indicators of the syringe augmentation device based on the determination.

16. The method of claim 15, wherein slidably receiving the syringe comprises:
slidably receiving a complete standard cylindrical chamber of the syringe in a compression sleeve housed within the attachment body of the syringe augmentation device; and
maintaining a position of the cylindrical chamber of the syringe within the compression sleeve as the syringe is compressed.

17. The method of claim 15, wherein selectively compressing the syringe comprises incrementally mechanically driving the plunger forward via a ratchet.

18. The method of claim 17, further comprising selectively adjusting the extent to which the plunger is driven forward in each increment.

19. The method of claim 15, wherein determining whether the measure pressure exceeds the predetermined threshold comprises:
first determining, by a controller having one or more processors, when the measured pressure exceeds a first predetermined threshold;
second determining, by the controller, when the measured pressure exceeds a second predetermined threshold; and
third determining, by the controller, when the measured pressure exceeds a third predetermined threshold.

20. The method of claim 19, wherein illuminating one or more feedback indicators comprises:
illuminating a first feedback indicator a first color based on the first determination;
illuminating a second feedback indicator a second color based on the second determination; and
illuminating a third feedback indicator a third color based on the third determination.
